(19) United States
(12) Patent Application Publication
(10) Pub. No.: US 2012/0021979 A1
(43) Pub. Date: Jan. 26, 2012

(54) GLP-1 receptor modulation of addiction, neuropsychiatric disorders and erectile dysfunction

(75) Inventors: Aurelio Galli, Brentwood, TN (US); Kevin Erreger, Nashville, TN (US); Kevin Niswender, Brentwood, TN (US); Sabrina Robertson, Durham, NC (US); Christine Saunders, Brentwood, TN (US); Heinrich J.G. Matthies, Nashville, TN (US); Adeola Davis, Mount Juliet, TN (US)

(73) Assignee: Vanderbilt University

(21) Appl. No.: 13/134,577

(22) Filed: Jun. 10, 2011

Related U.S. Application Data
(60) Provisional application No. 61/358,251, filed on Jun. 24, 2010.

(51) Int. Cl. A61K 38/26; A61P 15/10; A61P 3/10; A61P 25/30; A61K 38/16; A61K 31/4985

(52) U.S. Cl. 514/6.9; 514/11.7; 514/249

(57) ABSTRACT

The present invention involves the use of GLP-1 and its analogs or GLP-1 receptor agonists to modulate dopamine transporter signaling. The implications of this ability include the use of MT-1 and its analogs or GLP-1 receptor agonists, such as exendin-4, to blunt behavioral response to addictive drugs, to decrease drug dependence, to prevent drug abuse relapse, and to treat brain disorders such as neuropsychiatric disorders including ADHD. The present invention also involves the use of GLP-1 and its analogs or GLP-1 receptor agonists to activate GLP-1R in penile tissue, such as for the treatment of erectile dysfunction either as a monotherapy or in combinations with other treatments, such as PDE 5 inhibitors.

This application claims benefit of priority to U.S. Provisional Application Ser. No. 61/358,251, filed Jun. 24, 2010, the entire contents of which are hereby incorporated by reference.

This invention was made with government support under grant numbers R01DA014684-06A2, R01DK085712-0109, K99DA025716-01A109 and F31 MH084755-02 awarded by the National Institutes of Health. The government has certain rights in the invention.

### BACKGROUND OF THE INVENTION

A. Field of the Invention

In one aspect, the present invention relates generally to the fields of neurobiology and neurophysiology. More particularly, it concerns the use of glucagon-like peptide 1 (GLP-1) for modulating dopamine homeostasis and the activity of dopamine transporters in the context of addictive substance use and abuse and neuropsychiatric disorders.

In another aspect, the present invention relates generally to the fields of biology and pharmacology. More particularly, it concerns the use of GLP-1 receptor agonists, including GLP-1 and GLP-1 analogs, for treating erectile dysfunction.

B. Description of Related Art

1. Drug Dependence

It is estimated that 205 million people in the world use illicit drugs, including 25 million who suffer from illicit drug dependence. This constitutes a public health, socio-economic development and security problem of epic proportions in both industrialized and developing countries alike.

Drug dependence is considered a multi-factorial health disorder that often follows the course of a relapsing and remitting chronic disease. Unfortunately, in many societies, drug dependence is still not recognized as a health problem and many people suffering from it are stigmatized and have no access to treatment and rehabilitation. Over recent years, the biopsychosocial model has recognized drug dependence as a multifaceted problem requiring the expertise of many disciplines. A health sciences multidisciplinary approach can be applied to research, prevention and treatment.

Recent scientific evidence indicates that the development of dependence is a result of a complex multi-factorial interaction between repeated exposure to drugs, and biological and environmental factors. Attempts to treat and prevent drug use through tough penal sanctions for drug users fail because they do not take into account the neurological changes drug dependence has on motivation pathways in the brain.

Drug dependence and illicit drug use are associated with health problems, poverty, violence, criminal behavior, and social exclusion. Its total costs to society are difficult to estimate, but can certainly be measured in the hundreds of billions of dollars. In addition to the health care costs and other costs associated with the consequences of drug use; drug dependence involves also social costs in the form of loss of productivity and family income, violence, security problems, traffic and workplace accidents, and links with corruption. These result in overwhelming economic costs and an unacceptable waste of human resources.

Moreover, drug use, especially injecting drug use (IDU), is closely linked to HIV and hepatitis B and C transmission through the sharing of needles. Non-injecting drug use is also linked to HIV transmission by increasing high risk sexual behaviors. Data on the size of the IDU population from 130 countries indicates that there are close to 10 million IDUs worldwide. Up to 10% of global HIV infections are due to unsafe injecting drug use, and if Sub-Saharan Africa is excluded, up to 30% of global HIV infections are due to unsafe injecting drug use. The sharing of contaminated injection equipment is a major route of transmission in many regions, including Eastern Europe, Central, South and South Fast Asia and some countries in Latin America.

A number of forms of treatment for substance dependence exist, including pychotherapy and behavioral therapy, as well as replacement or antagonist drugs such as methadone, dextroamphetamine, clomethiazole, bromocriptine, desipramine, nalmefene, naltrexone, disulfiram, acamprosate, topiramate, modafinil, nocaine, vanoxerine, bupropion, mecamylamine, varenicline, lamotrigine, gabapentin, rimonabant, iobgaine, 18-methoxycoronaridine and memantine. Unfortunately, therapy for drug dependence does not have a high rate of long-term success, with relapse being common. Thus, improved methods of controlling, preventing and reducing dependence on additive substances is needed.

2. Erectile Dysfunction

Erectile dysfunction (ED) currently affects more than 18 million men in the U.S., with 35-50% of men having diabetes experiencing it. In diabetics, there is higher glucose in the blood, and this leads to hyperglycemia-induced increased glycosylation of eNOS, rendering it inactive by reciprocally decreasing phosphorylation at Ser-1177. This leads to less nitric oxide production, one of the main physiologic causes of ED. Current drugs on the market for ED are phosphodiesterase inhibitors, such as Viagra®. These drugs prevent degradation of cGMP, the moleculre responsible for activating the kinase called PKG. Activation of penile PKG leads to relaxation of smooth muscles during sexual stimulation thereby increasing blood flow. However, these drugs cannot be taken more than once a day and have side-effects associated with them. Other treatments for ED have their own limitations. Research on drugs for treating ED is expanding rapidly, and improved treatments are needed.

### SUMMARY OF THE INVENTION

Thus, in accordance with the present invention, there is provided a method of inhibiting a subject's response to an addictive substance comprising administering to said subject GLP-1 or analog thereof or an agonist of GLP-1 receptor. The subject may be a non-human mammal, such as a mouse, a rat, or a primate, or may be a human. Administering may comprise oral administration, intravenous administration, transdermal administration, or inhalation. The addictive substance may be alcohol, a narcotic, such as heroin or morphine, or a stimulant, such as amphetamine, 3,4-methylenedioxymethamphetamine, cocaine or methamphetamine. The subject may have been diagnosed with addictive behavior. The subject may currently be using an addictive substance or not currently using an addictive substance. The subject may further be receiving a second anti-addiction treatment.

In another embodiment, there is provided a method of treating a subject for addiction comprising administering to said subject GLP-1 or analog thereof or an agonist of GLP-1 receptor.

In yet another embodiment, there is provided a method of method of reducing a subject's dependence on an addictive substance comprising administering to said subject or analog thereof or an agonist of GLP-1 receptor.

In still yet another embodiment, there is provided a method of reducing a subject's chance of developing dependence on an addictive substance comprising administering to said subject or analog thereof or an agonist of GLP-1 receptor.

In yet a further embodiment, there is provided a method of modifying a subject's response to drug acting through the dopamine receptor comprising administering to said subject GLP-1 or analog thereof or an agonist of GLP-1 receptor.

An additional embodiment comprises a method of treating a subject with an addictive substance comprising (a) administering to said subject GLP-1 or analog thereof or an agonist of GLP-1 receptor; and (b) administering to said subject said addictive substance. The subject may be is a non-human mammal, such as a a mouse, a rat, or a non-human primate. The subject may instead be a human. Administering may comprise oral administration, intravenous administration, transdermal administration, or inhalation. The addictive substance may be alcohol, a narcotic, such as heroin or morphine, or a stimulant, such as amphetamine, 3,4-methylenedioxymethamphetamine, cocaine or methamphetamine.

Yet another embodiment comprises a method of treating erectile dysfunction (ED) in a male subject comprising administering to said subject GLP-1 or analog thereof or an agonist of GLP-1 receptor. The subject may be a mouse, a rat, or a non-human primate. The subject may be a human. More than one GLP-1 receptor agonist, GLP-1 and/or GLP-1 analog may be administered. Administering may comprises oral administration, intravenous administration, transdermal administration, or inhalation. The subject may have diabetes and/or cardiovascular disease. The method may further comprise administering to said subject a second ED therapy.

Still yet another embodiment includes a method of improving sexual performance in a male subject comprising administering to said subject GLP-1 or analog thereof or an agonist of GLP-1 receptor. The subject may be a mouse, a rat, or a non-human primate. The subject may he a human. More than one GLP-1 receptor agonist, GLP-1 and/or GLP-1 analog may he administered. Administering may comprises oral administration, intravenous administration, transdermal administration, or inhalation.

Yet an additional embodiment comprises a method of reducing one or more symptoms of erectile dysfunction (ED) in a male subject comprising administering to said subject GLP-1 or analog thereof or an agonist of GLP-1 receptor. The one or more symptoms may comprise partial erection, lack of erection, or limited erection duration. More than one GLP-1 receptor agonist may be administered, including GLP-1 and GLP-1 analog or multiple GLP-1 analogs. The administering may comprise oral administration, intravenous administration, transdermal administration, or inhalation.

A further embodiment includes a method of reducing one or more erectile dysfunction (ED) treatment side effects in a male subject comprising administering to said subject a reduced amount of ED treatment in combination with a GLP-1 or analog thereof or an agonist of GLP-1 receptor. The one or more treatment side effects may comprise priapism, vision abnormalities, hearing loss, headache, muscle ache, and dyspepsia. More, than one GLP-1 receptor agonist may be administered, including GLP-1 a GLP-1 analog, or multiple GLP-1 analogs. The administering may comprise oral administration, intravenous administration, transdermal administration, or inhalation.

Also provide is a method treating diabetes and erectile dysfunction (ED) in a male subject comprising administering to said subject an amount of a GLP-1R agonist effective to treat both diabetes and ED. More than one GLP-1 receptor agonist may be administered. The GLP-1 receptor agonist may be a GLP-1 analog. Administering may comprise oral administration, intravenous administration, transdermal administration, or inhalation. The method may further comprise administering to said subject a diabetes therapy.

As used herein the specification, “a” or “an” may mean one or more.

As used herein in the claim(s), when used in conjunction with the word “comprising,” the words “a” or “an” may mean one or more than one.

As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIG. 1—An example of GLP-1-induced increase in dopamine transporter surface expression. Representative immunoblots for surface and total fractions of dopamine transporter, tyrosine hydroxylase and Na+/K+ ATPase.

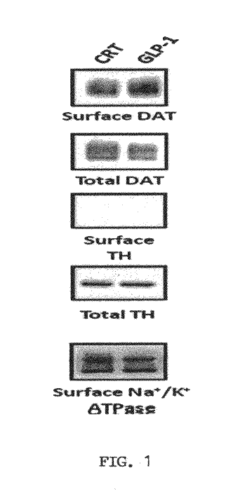

FIG. 2—Freshly homogenized rat tissue. Lanes left to right are lung, penis, stomach and pancreas. Bands show a Western blot for GLP-1R.

FIG. 3—Longitudinal Penile Section. Magnification is 40× (zoom 0.7). Green=GLP-1R; Red=nNOS; White=DIC.

FIGS. 4A-B—Penile Sections. (FIG. 4A) Longitudinal section. Magnification is 40× (zoom 0.7). Green=GLP-1R; Red=nNOS; Yellow→overlay. (FIG. 4B) Transverse section. Magnification is 40× (no zoom). Green=GLP-1R.

### DESCRIPTION OF ILLUSTRATIVE EMBODIMENTS

Here, the inventors have demonstrated, for the first time, effects of glucagon-like peptide 1 (GLP-1) on the dopamine transporter (DAT), dopamine homeostasis, and on the actions of the abused psychostimulant amphetamine. They show that GLP-1 regulates the cell surface levels of DAT in brain striatum, that GLP-1 modulates the release of DA induced by amphetamine from striatal brain slices, and that GLP-1 regulates dopamine transporter function an assay of dopamine clearance. Moreover, exendin-4 GLP-1 receptor agonist) reduces amphetamine-induced behaviors. These data provide the first evidence that dopaminergic signaling, noted for playing a key role in reward and addiction, may be modulated by molecules targeting the GLP-1 receptor. Moreover, GLP-1 receptor agonists like exendin-4 can blunt the behavioral response to drugs of abuse and affect reward systems.

ADHD is one of the most costly health conditions in the U.S. with annual cost of $77 billion. ADHD is diagnosed in approximately 3%-5% of the school-age population. About 30%-80% of children with the disorder will continue to have ADHD symptoms into adolescence. Studies point to disruption of the dopamine system as underlying ADHD. Consistent with the ability of GLP-1 to increase dopamine transporter cell surface expression and to blunt locomotion, these data implicate GLP-1 has a putative therapeutic agent for ADHD. The results further have therapeutic implications for both diabetes and obesity, which affect 2 in 3 adults in the United States.

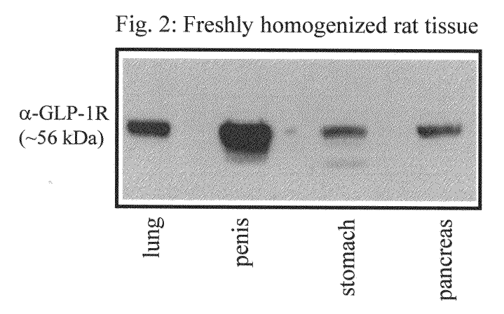

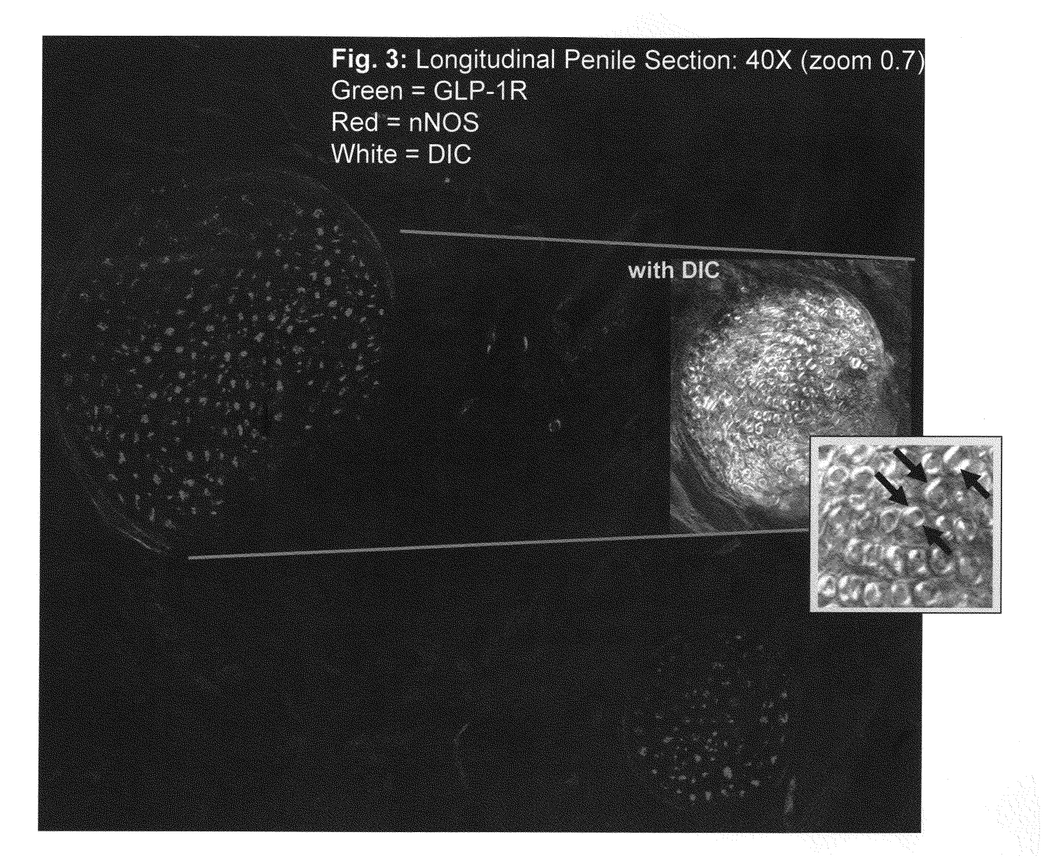

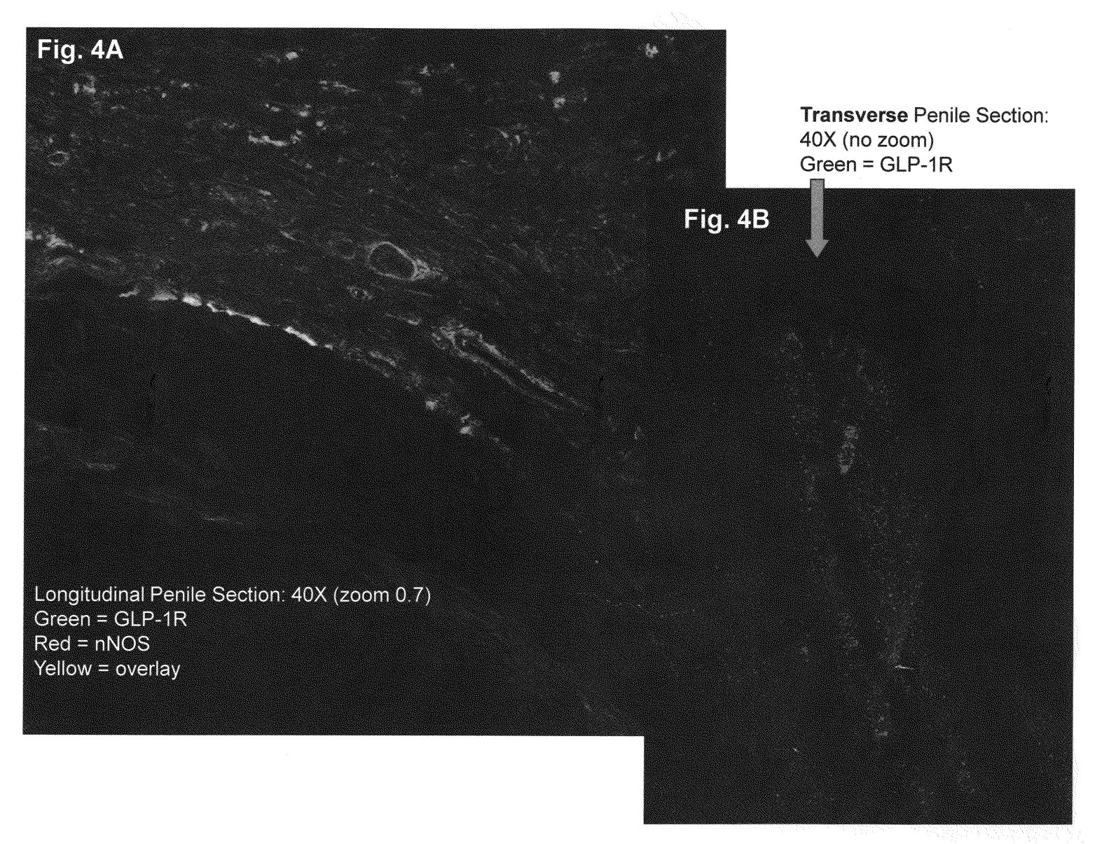

Also, recent studies have shown that the effects of the GLP-1 receptor (GLP-1R) in gastric, myocardial and pulmonary arterial tissue are through a NO-dependent mechanism (presumably via a Giα2, p38 MAP kinase-dependent mechanism, since p38 MAP kinase activation is a potent stimulator of NOS2 activity). Here, the inventors have demonstrated, for the first time, the presence of the GLP-1R in the penis, thereby opening the possibility of using GLP-1 as an ED therapy. This was demonstrated by both Western blotting (FIG. 2) and immunohistochemistry coupled with confocal microscopy (FIGS. 3 and 4A-B).

The significance of this finding is the potential for treatments of ED targeting penile GLP-1R, both in normal and diabetic patients. GLP-1R agonists and GLP-1 analogs lower blood glucose in diabetics. If these drugs are given orally or administered locally to patients with ED, activation of the penile GLP-1R would increase blood flow to the penis, and potentially serve as a novel therapeutic approach for the treatment of ED. The side-effects or GLP-1 receptor agonists and GLP-1 analogs as used as anti-diabetes drugs are limited. Thus, the implications of this finding are potentially far-reaching.

These and other aspects of the invention are described in detail below.

### A. DOPAMINE TRANSPORTER (DAT)

The dopamine transporter (DAT) is a member of the subfamily of monoamine transporters with numerous common topological structures and significant amino acid sequence homology. DAT has been identified as located on the distal end of chromosome 5 (5p15.3) (Giros et al., 1992). Kawarai et al. (1997), isolated and characterized the human DAT gene (hDAT) including about 1 kb of 5′-flanking region. The hDAT gene spans over 64 kb, consisting of 15 exons separated by 14 introns. The intron-exon structure of the hDAT gene is most similar to that of the human NET gene.

Promoter sequence analysis demonstrated a ‘TATA’-less, ‘CAT’-less and G+C-rich structure. Two F box and several Sp-1-binding sites exist in the promoter region. These structural features are similar to that of the human D1A dopamine receptor gene and the human monoamine oxidase A gene. The DAT gene encodes for a 620-amino acid protein with a calculated molecular weight of 68,517 (Giros et al., 1992) and is associated with numerous neuropsychiatric disorders (Bannon, 2001). Examples of neurological diseases involving dopamine transporter function include schizophrenia, addiction disorders, attention deficit hyperactivity disorder (ADHD), psychoses, Tourette's syndrome, and Parkinson's disease.

### B. ADDICTION AND ADDICTIVE SUBSTANCES

1. Addiction

In medicine, an addiction is a chronic neurobiological disorder that has genetic, psychosocial, and environmental dimensions and is characterized by one of the following: the continued use of a substance despite its detrimental effects, impaired control over the use of a substance (compulsive behavior), and preoccupation with a substance use for non-therapeutic purposes (i.e., craving the drug).

Tolerance to a drug and physical dependence are not defining characteristics of addiction, although they typically accompany addiction to certain drugs. Tolerance is a pharmacologic phenomenon where the dose of a medication needs to he continually increased in order to maintain its desired effects. For instance, individuals with severe chronic pain taking opiate medications (like morphine) will need to continually increase the dose in order to maintain the drug's analgesic (pain-relieving) effects. Physical dependence is also a pharmacologic property and means that if a certain drug is abruptly discontinued, an individual will experience certain characteristic withdrawal signs and symptoms. Many drugs used for therapeutic purposes produce withdrawal symptoms when abruptly stopped, for instance oral steroids, certain antidepressants, benzodiazepines, and opiates.

Pharmacologists continue to speak of addiction from a physiologic standpoint (some call this a physical dependence); psychiatrists refer to the disease state as psychological dependence; most other physicians refer to the disease as addiction. The medical community now makes a careful theoretical distinction between physical dependence (characterized by symptoms of withdrawal) and psychological dependence (or simply addiction). Addiction is now narrowly defined as “uncontrolled, compulsive use”; if there is no harm being suffered by, or damage done to, the patient or another party, then clinically it may he considered compulsive, but to the definition of some it is not categorized as ‘addiction.’ In practice, the two kinds of addiction are not always easy to distinguish. Addictions often have both physical and psychological components.

There is also a lesser known situation called pseudo-addiction. A patient will exhibit drug-seeking behavior reminiscent of psychological addiction, but they tend to have genuine pain or other symptoms that have been under-treated. Unlike true psychological addiction, these behaviors tend to stop when the pain is adequately treated. The obsolete term physical addiction is deprecated, because of its connotations. In modern pain management with opioids physical dependence is nearly universal. While opiates are essential in the treatment of acute pain, the benefit of this class of medication in chronic pain is not well proven. Clearly, there are those who would not function well without opiate treatment; on the other hand, many states are noting significant increases in non-intentional deaths related to opiate use. High-quality, long-term studies are needed to better delineate the risks and benefits of chronic opiate use.

Physical dependence on a substance is defined by the appearance of characteristic withdrawal symptoms when the substance is suddenly discontinued. Opiates, benzodiazepines, barbiturates, alcohol and nicotine induce physical dependence. On the other hand, some categories of substances share this property and are still not considered addictive: cortisone, β blockers and most antidepressants are examples. So, while physical dependency can be a major factor in the psychology of addiction and most often becomes a primary motivator in the continuation of an addiction, the initial primary attribution of an addictive substance is usually its ability to induce pleasure, although with continued use the goal is not so much to induce pleasure as it is to relieve the anxiety caused by the absence of a given addictive substance, causing it to become used compulsively.

Some substances induce physical dependence or physiological tolerance—but not addiction—for example many laxatives, which are not psychoactive; nasal decongestants, which can cause rebound congestion if used for more than a few days in a row; and some antidepressants, most notably venlafaxine, paroxetine and sertraline, as they have quite short half-lives, so stopping them abruptly causes a more rapid change in the neurotransmitter balance in the brain than many other antidepressants. Many non-addictive prescription drugs should not be suddenly stopped, so a doctor should be consulted before abruptly discontinuing them.

The speed with which a given individual becomes addicted to various substances varies with the substance, the frequency of use, the means of ingestion, the intensity of pleasure or euphoria, and the individual's genetic and psychological susceptibility. Some people may exhibit alcoholic tendencies from the moment of first intoxication, while most people can drink socially without ever becoming addicted. Opioid dependent individuals have different responses to even low doses of opioids than the majority of people, although this may be due to a variety of other factors, as opioid use heavily stimulates pleasure-inducing neurotransmitters in the brain. Nonetheless, because of these variations, in addition to the adoption and twin studies that have been well replicated, much of the medical community is satisfied that addiction is in part genetically moderated. That is, one's genetic makeup may regulate how susceptible one is to a substance and how easily one may become psychologically attached to a pleasurable routine.

The DSM definition of addiction can be boiled down to compulsive use of a substance (or engagement in an activity) despite ongoing negative consequences—this is also a summary of what used to be called “psychological dependency.” Physical dependence, on the other hand, is simply needing a substance to function. Humans are all physically dependent on oxygen, food and water. A drug can cause physical dependence and not addiction (for example, some blood pressure medications, which can produce fatal withdrawal symptoms if not tapered) and can cause addiction without physical dependence (the withdrawal symptoms associated with cocaine are all psychological, there is no associated vomiting or diarrhea as there is with opioid withdrawal).

In the now outdated conceptualization of the problem, psychological dependency leads to psychological withdrawal symptoms (such as cravings, irritability, insomnia, depression, anorexia, etc.). Addiction can in theory he derived from any rewarding behaviour, and is believed to be strongly associated with the dopaminergic system of the brain's reward system (as in the case of cocaine and amphetamines). Some claim that it is a habitual means to avoid undesired activity, but typically it is only so to a clinical level in individuals who have emotional, social, or psychological dysfunctions (psychological addiction is defined as such), replacing normal positive stimuli not otherwise attained.

A person who is physically dependent, but not psychologically dependent can have their dose slowly dropped until they are no longer physically dependent. However, if that person is psychologically dependent, they are still at serious risk for relapse into abuse and subsequent physical dependence.

Psychological dependence does not have to be limited only to substances; even activities and behavioural patterns can be considered addictions, if they become uncontrollable. e.g., problem gambling, Internet addiction, computer addiction, sexual addiction/pornography addiction, overeating, self-injury, compulsive buying, or work addiction.

The American Society of Addiction Medicine recommends treatment for people with chemical dependency based on patient placement criteria (currently listed in PPC-2), which attempt to match levels of care according to clinical assessments in six areas, including:
acute intoxication and/or withdrawal potential
biomedical conditions or complications
emotional/behavioral conditions or complications
treatment acceptance/resistance
relapse potential
recovery environment
   Some medical systems, including those of at least 15 states of the United States, refer to an Addiction Severity Index to assess the severity of problems related to substance use. The index assesses problems in six areas: medical, employment/support, alcohol and other drug use, legal, family/social, and psychiatric.

While addiction or dependency is related to seemingly uncontrollable urges, and arguably could have roots in genetic predispositions, treatment of dependency is conducted by a wide range of medical and allied professionals, including Addiction Medicine specialists, psychiatrists, psychologists, and appropriately trained nurses, social workers, and counselors. Early treatment of acute withdrawal often includes medical detoxification, which can include doses of anxiolytics or narcotics to reduce symptoms of withdrawal. An experimental drug, ibogaine, is also proposed to treat withdrawal and craving. Alternatives to medical detoxification include acupuncture detoxification. A wide variety of controlled clinical trials, outcome summaries and anecdotal reports about the use of acupuncture in addiction treatment have been appearing since the 1970s in journals specializing in addictions, mental health, public health, criminal justice and acupuncture. These reports differed vastly in terms of methodology, populations studied, statistical sophistication and clinical relevance as well as in their findings about the value of acupuncture. A sub-category of this published work has focused specifically on acupuncture detoxification. Neurofeedback therapy has shown statistically significant improvements in numerous researches conducted on alcoholic as well as mixed substance abuse population. In chronic opiate addiction, a surrogate drug such as methadone is sometimes offered as a form of opiate replacement therapy. But treatment approaches universal focus on the individual's ultimate choice to pursue an alternate course of action. Therapists often classify patients with chemical dependencies as either interested or not interested in changing. Treatments usually involve planning for specific ways to avoid the addictive stimulus, and therapeutic interventions intended to help a client learn healthier ways to find satisfaction. Clinical leaders in recent years have attempted to tailor intervention approaches to specific influences that affect addictive behavior, using therapeutic interviews in an effort to discover factors that led a person to embrace unhealthy, addictive sources of pleasure or relief from pain.

2. Addictive Substances

A wide variety of substances are considered potentially addictive. Perhaps the most well known is alcohol. About 12% of American adults have had an alcohol dependence problem at some time in their life. Alcohol dependence is acknowledged by the American Medical Association as a disease because it has a characteristic set of signs and symptoms and a progressive course. It has significant socio-economic impact and remains a challenge to treat.

Opioid dependence is a medical diagnosis characterized by an individual's inability to stop using opioids even when objectively it is in his or her best interest to do so. In 1964, the WI-10 introduced “dependence” as a cluster of physiological, behavioral and cognitive phenomena of variable intensity, in which the use of drug (or drugs) takes on a high priority. The necessary descriptive characteristics are preoccupation with a desire to obtain and take the drug and persistent drug-seeking behavior. Opiates include the natural opiates (alkaloids contained in the resin of the opium poppy, primarily morphine, codeine, and thebaine; also, the leaves from Mitragyna Speciosa (also known as Kratom), and Salvinorin A). Semi-synthetic opioids include those created from natural opiates, such as hydromorphone, hydrocodone, oxycodone, oxymorphone, desomorphine, diacetylmorphine (heroin), nicomorphine, dipropanoylmorphine, benzylmorphine and ethylmorphine and buprenorphine. Fully synthetic opioids include fentanyl, pethidine, methadone, tramadol and dextropropoxyphene. Endogenous opioid peptides include endorphins, enkephalins, dynorphins, and endomorphins.

Amphetamine dependence (or amphetamine-like) refers to a state of dependence on a drug in the amphetamine class. Tolerance is developed rapidly in amphetamine abuse, thereby the amount of the drug that is needed to satisfy the addiction needs to he increased at regular intervals. Many users will repeat the amphetamine cycle by taking more of the drug during the withdrawal. This leads to a very dangerous cycle and may involve the use of other drugs to get over the withdrawal process. Users will commonly stay up for 2 or 3 days to avoid the withdrawals then dose themselves with benzodiazepines, barbiturate, and in some cases heroin, to help them stay calm while they recuperate or simply to extend the positive effects of the drug. Chronic abusers of amphetamine often insufflate, smoke or inject the drug in order to experience a rush, with extra risks for infection, vein damage and overdose.

Amphetamine is a chiral compound. The racemic mixture can be divided into its optical isomers: levo- and dextro-amphetamine. Amphetamine is the parent compound of its own structural class, comprising a broad range of psychoactive derivatives, from empathogens, MDA (3,4-Methylenedioxyamphetamine) and MDMA (3,4-Methylenedioxy-N-methamphetamine) known as ecstasy, to the N-methylated form, methamphetamine known as “meth,” and to decongestants such as ephedrine (EPI-1). Amphetamine is a homologue of phenethylamine.

Sedatives, hypnotics, or anxiolytics (including benzodiazepines and barbiturates) are drugs of the psychoactive class whose primary function is to induce sleep and to he used in the treatment of insomnia and in surgical anesthesia. When used in anesthesia to produce and maintain unconsciousness, “sleep” is metaphorical and there are no regular sleep stages or cyclical natural states when used in anesthesia; patients rarely recover from anesthesia feeling refreshed and with renewed energy. Because drugs in this class generally produce dose-dependent effects, ranging from anxiolysis to production of unconsciousness, they are often referred to collectively as sedative-hypnotic drugs.

Hypnotic drugs are regularly prescribed for insomnia and other sleep disorders, with over 95% of insomnia patients being prescribed hypnotics in some countries. However, many hypnotic drugs are habit-forming. Hypnotic medication when prescribed should he used for the shortest period of time possible. In addition, the benzodiazepine and nonhenzodiazepine hypnotic medications also have a number of side effects such as daytime fatigue, motor vehicle crashes, cognitive impairments and falls and fractures. Gradual discontinuation of hypnotics leads to improved health without worsening of sleep. Preferably they should be prescribed for only a few days at the lowest effective dose and avoided altogether wherever possible in the elderly.

Other additictive substance prone to abuse include cocaine, marijuana, hallucinogens (LSD), inhalants, phencyclidine (or phencyclidine-like) and nicotine.

3. Treatments

Treatment for drug abuse or dependence begins with recognizing the problem. Though “denial” used to be considered a symptom of addiction, recent research has shown that people who are addicted have far less denial if they are treated with empathy and respect, rather than told what to do or “confronted.”

Treatment of drug dependency involves stopping drug use either gradually or abruptly (detoxification), support, and staying drug free (abstinence). People with acute intoxication or drug overdose may need emergency treatment. Sometimes, the person loses consciousness and might need to be on a breathing machine (mechanical respirator) temporarily. The treatment depends on the drug being used.

Detoxification is the withdrawal of an abused substance in a controlled environment. Sometimes a drug with a similar action is taken instead, to reduce the side effects and risks of withdrawal. Detoxification can be done on an inpatient or outpatient basis. Palliative treatments may ease the discomfort of withdrawal.

Residential treatment programs monitor and address possible withdrawal symptoms and behaviors. These programs use behavior modification techniques, which are designed to get users to recognize their behaviors.

For narcotic dependence, some people are treated with methadone or similar drugs to prevent withdrawal and abuse. The goal is to enable the person to live as normal a life as possible.

### C. ERECTILE DYSFUNCTION

Erectile dysfunction (ED, or “male impotence”) is a sexual dysfunction characterized by the inability to develop or maintain an erection of the penis sufficient for satisfactory sexual performance. An erection occurs as a hydraulic effect due to blood entering and being retained in sponge-like bodies within the penis. The process is most often initiated as a result of sexual arousal, when signals are transmitted from the brain to nerves in the penis.

Penile erection is managed by two different mechanisms. The first one is the reflex erection, which is achieved by directly touching the penile shaft. The second is the psychogenic erection, which is achieved by erotic or emotional stimuli. The former uses the peripheral nerves and the lower parts of the spinal cord, whereas the latter uses the limbic system of the brain. In both conditions, an intact neural system is required for a successful and complete erection. Stimulation of penile shaft by the nervous system leads to the secretion of nitric oxide (NO), which causes the relaxation of smooth muscles of corpora cavernosa (the main erectile tissue of penis), and subsequently penile erection. Additionally, adequate levels of testosterone (produced by the testes) and an intact pituitary gland are required for the development of a healthy erectile system.

As can be understood from the mechanisms of a normal erection, impotence may develop due to hormonal deficiency, disorders of the neural system, lack of adequate penile blood supply or psychological problems. Restriction of blood flow can arise from impaired endothelial function due to the usual causes associated with coronary artery disease, but can also be caused by prolonged exposure to bright light.

Erectile dysfunction is indicated when an erection is difficult to produce. There are various circulatory causes, including alteration of the voltage-gated potassium channel as in the case of arsenic poisoning from drinking water. The most important organic causes are cardiovascular disease and diabetes, neurological problems (spinal cord and brain injuries, nerve disorders such as Parkinson's disease. Alzheimer's disease, multiple sclerosis, and stroke, trauma from prostatectomy surgery), hormonal insufficiencies (hypogonadism), kidney failure, aging and drug side effects (such as anti-depressants and nicotine).

Psychological impotence is where erection or penetration fails due to thoughts or feelings (psychological reasons) rather than physical impossibility; this is somewhat less frequent but often can be helped. Notably in psychological impotence, there is a strong response to placebo treatment. Erection, tied closely as it is about ideas of physical well being, can have severe psychological consequences.

Besides treating the underlying causes such as potassium deficiency or arsenic contamination of drinking water, the first line treatment of erectile dysfunction consists of a trial of PDE5 inhibitor drugs (the first of which was sildenafil or Viagra®). In some cases, treatment can involve prostaglandin tablets in the urethra, intracavernous injections with a fine needle into the penis that cause swelling, a penile prosthesis, a penis pump or vascular reconstructive surgery.

Surgical intervention for a number of different conditions may remove anatomical structures necessary to erection, damage nerves, or impair blood supply. Complete removal of the prostate gland or external beam radiotherapy of the gland are common causes of impotence; both are treatments for prostate cancer. Some studies have shown that male circumcision may result in an increased risk of impotence, while others have found no such effect.

1. Diagnosis

There are no formal tests to diagnose erectile dysfunction. Some blood tests arc generally done to exclude underlying disease, such as hypogonadism and prolactinoma. Diabetes is considered a disorder, but is also a risk. Impotence is also related to generally poor physical health, poor dietary habits, obesity, and most specifically cardiovascular disease such as coronary artery disease and peripheral vascular disease.

A useful and simple way to distinguish between physiological and psychological impotence is to determine whether the patient ever has an erection. If never, the problem is likely to be physiological. If sometimes (however rarely), it could be physiological or psychological. The current diagnostic and statistical manual of mental diseases includes duplex ultrasound, penile nerve function, nocturnal penile tumescence (NPT) testing, penile biothesiometry, dynamic infusion cavernosometry (MCC), corpus cavemosometry, digital subtraction angiography (DSA), and magnetic resonance angiography (MRA).

2. Treatments

Treatment depends on the cause. Testosterone supplements may he used for cases due to hormonal deficiency. However, the cause is more usually lack of adequate penile blood supply as a result of damage to inner walls of blood vessels. This damage is more frequent in older men, and often associated with disease, in particular diabetes.

Treatments (with the exception of testosterone supplementation, where effective) work on a temporary basis: they enable an erection to be attained and maintained long enough for intercourse, but do not permanently improve the underlying condition.

ED can in many cases be treated by drugs taken orally, injected, or as penile suppositories. These drugs increase the efficacy of nitric oxide, which dilates the blood vessels of corpora cavernosa.

Exercise, particularly aerobic exercise is an effective cheap treatment for erectile dysfunction. Another non-pharmacologic method involves a purpose-designed external vacuum pump can he used to attain erection, with a separate compression ring fitted to the penis to maintain it. More drastically, inflatable or rigid penile implants may be fitted surgically. All these mechanical methods are based on simple principles of hydraulics and mechanics and are quite reliable, but have their disadvantages. Intracavernous pharmacotherapy is also used by men with certain medical conditions such as hypertension or high blood pressure.

The cyclic nucleotide phosphodiesterases constitute a group of enzymes that catalyse the hydrolysis of the cyclic nucleotides cyclic AMP and cyclic GMP. They exist in different molecular forms and are unevenly distributed throughout the body. One of the forms of phosphodiesterase is termed PDE5. The prescription PDE5 inhibitors sildenafil (Viagra®), vardenafil (Levitra®) and tadalafil (Cialis®) are prescription drugs which are taken orally. They work by blocking the action of PDE5, which causes cGMP to degrade.

Alprostadil has become available in some countries as a topical cream (under the brand name Befar®), and preliminary studies have shown a clinical efficacy of up to 83%.

### D. THERAPEUTIC COMPOSITIONS AND METHODS

The present invention provides new methods for treating drug dependence/addiction. Subjects suffering from dependence/addiction include individuals addicted to a variety of agents, such as narcotics and amphetamines. They addition may also be a compulsion, such as some version of obsessive-compulsive disorder (excessive cleanliness, hoarding, over-eating etc.). Following identification of the patient as sufferring from addiction or OCD, a GLP-1R modulator will be selected based on the particular conditions of the patient, including but not limited a consideration of other drugs that the patient may be taking.

The present invention also provides new methods for treating ED. Subjects suffering from ED include a variety of individuals, ranging from those having cardiovascular disease and diabetes, neurological problems, hormonal insufficiencies, kidney failure, aging and drug side effects. As with drug dependence treatments, following identification of the patient as suffering from ED, a GLP-1R modulator will be selected based on the particular conditions of the patient, including but not limited a consideration of other drugs that the patient may be taking.

1. GLP-1

Glucagon-like peptide-1 (GLP-1) is derived from the transcription product of the proglucagon gene. The major source of GLP-1 in the periphery is the intestinal L cell that secretes GLP-1 as a gut hormone. The biologically active forms of GLP-1 are: GLP-1-(7-37) and GLP-1-(7-36)-NH₂. GLP-1 is also a neurotransmitter synthesized by a small population of neurons in the nucleus of the solitary tract (NTS) in the caudal brainstem. These neurons project widely into the brain and putatively release GLP-1. The GLP-1R is expressed throughout the brain and the relative contributions of central (brain) and peripheral both GLP-1 peptide sources and receptor signaling are unresolved.

GLP-1 secretion by L cells is dependent on the presence of nutrients in the lumen of the small intestine. The secretagogues (agents that cause or stimulate secretion) of this hormone include major nutrients like carbohydrate, protein and lipid. Once in the circulation. GLP-1 has a half life of less than 2 minutes, due to rapid degradation by the enzyme dipeptidyl peptidase-4.

GLP-1 possesses several physiological properties that make it (and its analogs) a subject of intensive investigation as a potential treatment of diabetes mellitus. The known physiological functions of GLP-1 include (a) increasing insulin secretion from the pancreas in a glucose-dependent manner; (b) decreasing glucagon secretion from the pancreas; (c) increasing β cell mass and insulin gene expression; (d) inhibiting acid secretion and gastric emptying in the stomach; (e) decreasing food intake by increasing satiety; and (f) promoting insulin sensitivity

2. GLP-1 Receptor Agonists

Glucagon-like peptide-1 analogs are a new class of drug for treatment of type 2 diabetes. One of their advantages is that they have a lower risk of causing hypoglycemia. Exenatide (2005) and liraglutide (2010) are approved, and albiglutide and taspoglutide remain under evaluation.

Exenatide. Marketed as Byetta®, exenatide is approved for the treatment of diabetes mellitus type 2. Exenatide is administered as a subcutaneous injection (under the skin) of the abdomen, thigh, or arm, 30 to 60 minutes before the first and last meal of the day. Exenatide is a synthetic version of exendin-4, a hormone found in the saliva of the Gila monster. It displays biological properties similar to human glucagon-like peptide-1 (GLP-1), a regulator of glucose metabolism and insulin secretion. According to the package insert, exenatide enhances glucose-dependent insulin secretion by the pancreatic beta-cell, suppresses inappropriately elevated glucagon secretion, and slows gastric emptying, although the mechanism of action is still under study.

Exenatide is a 39-amino-acid peptide that is an insulin secretagogue with glucoregulatory effects. Exenatide was approved by the FDA in 2005 for patients whose diabetes was not well-controlled on other oral medication. The medication is injected subcutaneously twice per day using a pre-filled pen device. The abdomen is a common injection site, after the area is cleaned with an alcohol pad. A new pen must first be tested to see if the medicine is flowing

The incretin hormones GLP-1 and glucose-dependent insulinotropic peptide (GIP) are produced by the L and K endocrine cells of the intestine following ingestion of food. GLP-1 and GIP stimulate insulin secretion from the β cells of the islets of Langerhans in the pancreas. Only GLP-1 causes insulin secretion in the diabetic state; however; GLP-1 itself is ineffective as a clinical treatment for diabetes as it has a very short half-life in vivo. Exenatide hears a 50% amino acid homology to GLP-1 and it has a longer half-life in vivo. Thus, it was tested for its ability to stimulate insulin secretion and lower blood glucose in mammals and was found to be effective in the diabetic state. In studies on rodents it has also been shown to increase the number of beta cells in the pancreas.

Exenatide is approved “as adjunctive therapy to improve glycemic control in patients with type 2 diabetes mellitus who are taking metformin, a biguanide, or a combination of metformin and a sulfonylurea but have not achieved adequate glycemic control.” It has now been approved for use with thiazolidinediones such as pioglitazone or rosiglitazone.

Exenatide raises insulin levels quickly (within about ten minutes of administration) with the insulin levels subsiding substantially over the next hour or two. A dose taken after meals has a much smaller effect on blood sugar than one taken beforehand. The effects on blood sugar diminish after 6-8 hours. The medicine is available in two doses: 5 mcg and 10 mcg. Treatment often begins with the 5 mcg dosage, which is increased if adverse effects are not significant.

According to the manufacturer, the exenatide (Byetta®) autoinjector must he stored in a refrigerator between 36° F. (2° C.) and 46° F. (8° C.) before first use, and then at a temperature between 36° F. (2° C.) and 77° F. (25° C.). In hot weather, therefore, they should be refrigerated. Exenatide (Byetta®) pens contain sixty doses designed to be used twice a day for 30 days.

U.S. Pat. No. 5,424,286 describes Exenatide and is hereby incorporated by reference.

Exenatide is believed to facilitate glucose control in at least five ways. Exenatide augments pancreas response (i.e., increases insulin secretion) in response to eating meals; exenatide suppresses pancreatic release of glucagon in response to eating, which helps stop the liver from overproducing sugar when it is unneeded, which prevents hyperglycemia (high blood sugar levels); exenatide helps slow down gastric emptying and thus decreases the rate at which meal-derived glucose appears in the bloodstream; exenatide has a subtle yet prolonged effect to reduce appetite, promote satiety via hypothalamic receptors (different receptors than for amylin); and exenatide reduces liver fat content.

The main side effects of exenatide use are gastrointestinal in nature, including acid or sour stomach, belching, diarrhea, heartburn, indigestion, nausea, and vomiting; exenatide is therefore not meant for people with severe gastrointestinal disease. Other side effects include dizziness, headache, and feeling jittery. Drug interactions listed on the package insert include delayed or reduced concentrations of Lovastatin, Paracetamol (Acetaminophen), and Digoxin, although this has not been proven to alter the effectiveness of these other medications.

Liraglutide. Liraglutide, marketed under the brand name Victoza®, is a long-acting glucagon-like peptide-1 (GLP-1) analog that has been developed for the treatment of type 2 diabetes. The product was approved by the European Medicines Agency (EMEA) in 2009, and by the U.S. Food and Drug Administration (FDA) in 2010.

Studies to date suggest liraglutide improves control of blood glucose. It reduces meal-related hyperglycemia (for 12 hours after administration) by increasing insulin secretion, delaying gastric emptying, and suppressing prandial glucagon secretion. Liraglutide may have advantages over current therapies: (a) it acts in a glucose-dependent manner, meaning that it will stimulate insulin secretion only when blood glucose levels are higher than normal, and consequently, it shows negligible risk of hypoglycemia; (b) it has the potential for inhibiting apoptosis and stimulating regeneration of beta cells (seen in animal studies); (c) it decreases appetite and maintains body weight, as shown in a head-to-head study versus glimepiride; (d) it lowers blood triglyceride levels; and (e) it has only mild and transient side effects, mainly gastrointestinal.

Liraglutide is a once-daily GLP-1 derivative for the treatment of type 2 diabetes. GLP-1, in its natural form, is short-lived in the body (the half-life after subcutaneous injection is approximately one hour), so it is not very useful as a therapeutic agent. However, liraglutide has a half-life after subcutaneous injection of 11-15 hours, making it suitable for once-daily dosing (which is less frequent than the currently approved Byetta form of exenatide which is twice daily, but is considerably more frequent than the once weekly form of exenatide that is scheduled for a decision from the FDA regarding marketing approval by early March 2010).

The prolonged action of liraglutide is achieved by attaching a fatty acid molecule at one position of the GLP-1 molecule, enabling it to bind to albumin within the subcutaneous tissue and bloodstream. The active GLP-1 is then released from albumin at a slow, consistent rate. Binding with albumin also results in slower degradation and reduced elimination of liraglutide from the circulation by the kidneys compared to GLP-1.

Sitagliptin. Sitagliptin (trade name Januvia®) is an oral antihyperglycemic (anti-diabetic drug) of the dipeptidyl peptidase-4 (DPP-4) inhibitor class, Sitagliptin being the only second generation DPP-4 inhibitor currently available in the USA. This enzyme-inhibiting drug is used either alone or in combination with other oral antihyperglycemic agents (such as metformin or a thiazolidinedione) for treatment of diabetes mellitus type 2. The benefit of this medicine is its lower side-effects (e.g., less hypoglycemia, less weight gain) in the control of blood glucose values. Exenatide (Byetta®) also works by its effect on the incretin system.

In clinical trials, adverse effects were as common with sitagliptin (whether used alone or with metformin or pioglitazone) as they were with placebo, except for extremely rare nausea and common cold-like symptoms. There is no significant difference in the occurrence of hypoglycemia between placebo and sitagliptin.

The DPP-4 enzyme is known to be involved in the suppression of certain malignancies, particularly in limiting the tissue invasion of these tumours. Inhibiting the DPP-4 enzymes may allow some cancers to progress. A study of DPP-4 inhibition in human non-small cell lung cancer (NSCLC) concluded that “DPPIV functions as a tumor suppressor, and its downregulation may contribute to the loss of growth control in NSCLC cells.” The risk of cancer suppression with DPP-4 down-regulation applies to all the DPP-4 inhibitors on the market in addition to sitagliptin (saxagliptin and vildagliptin) Sitagliptin works to competitively inhibit the enzyme dipeptidyl peptidase 4 (DPP-4). This enzyme breaks down the incretins GLP-1 and GIP, gastrointestinal hormones that are released in response to a meal. By preventing GLP-1 and GIP inactivation, GLP-1 and GIP are able to potentiate the secretion of insulin and suppress the release of glucagon by the pancreas. This drives blood glucose levels towards normal. As the blood glucose level approaches normal, the amounts of insulin released and glucagon suppressed diminishes thus tending to prevent an “overshoot” and subsequent low blood sugar (hypoglycemia) which is seen with some other oral hypoglycemic agents.

3. Pharmaceutical Formulations

It will he necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when compositions are introduced into a patient. Aqueous compositions of the present invention comprise an effective amount of the substance to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will he via any common route so long as the target tissue is available via that route. Such routes include oral, nasal, buccal, rectal, vaginal or topical route. Alternatively, administration may be by orthotopic, intradermal, subcutaneous, intramuscular, intraperitoneal, or intravenous injection. Such compositions would normally he administered as pharmaceutically acceptable compositions, described supra.

The active compounds may also be administered parenterally or intraperitoneally. Solutions of the active compounds as free base or pharmacologically acceptable salts can he prepared in water suitably mixed with a surfactant, such as hydroxypropylcellulose. Dispersions can also he prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must he sterile and must he fluid to the extent that easy syringability exists. It must be stable under the conditions of manufacture and storage and must he preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will he preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic. tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should he suitably buffered if necessary and the liquid diluent first rendered isotonic with sufficient saline or glucose. These particular aqueous solutions arc especially suitable for intravenous, intramuscular, subcutaneous and intraperitoneal administration. In this connection, sterile aqueous media which can be employed will be known to those of skill in the art in light of the present disclosure. For example, one dosage could he dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose For the individual subject. Moreover, for human administration, preparations should meet sterility, pyrogenicity, general safety and purity standards as required by FDA Office of Biologics standards.

4. Combinations

In some modalities, the compounds of the present invention are provided in combination with a second agent or therapy—either an anti-addictive substance/therapy or an agent designed to eliminate to reduce addiction, or to treat ED. These therapies may be provided together, i.e., administering both agents/therapies at the same time. This may he achieved by administering a single composition or pharmacological formulation that includes both agents, or by administering two distinct compositions or formulations, at the same time, wherein one composition includes the GLP-1 agonist and the other includes the second agent.

Alternatively, the GLP-1 treatment may precede or follow the second agent by intervals ranging from minutes to weeks. In embodiments where the second agent and the GLP-1 treatment are made separately, one would generally ensure that a significant period of time did not expire between the time of each delivery, such that the GLP-1 therapy would still he able to exert an advantageous effect on the subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1. 2, 3, 4, 5, 6, 7 or 8) lapse between the respective administrations.

It also is conceivable that more than one administration of either the GLP-1 treatment and/or the second therapy will be desired. Various combinations may be employed, where the GLP-1 treatment is “A,” and the second agent is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

A second agent/therapy for treating addiction includes counseling, an palliative agent or therapy or a addictive agent substitute, such a methadone.

A second agent for ED therapy includes inhibitors of cyclic nucleotide phosphodiesterases (PDEs), which constitute a group of enzymes that catalyse the hydrolysis of the cyclic nucleotides cyclic AMP and cyclic GMP. In particular, PDE5 inhibitors sildenafil (Viagra®), vardenafil (Levitra®) and tadalafil (Cialis®) are prescription drugs which are taken orally. They work by blocking the action of PDE5, which causes cGMP to degrade. Alprostadil has become available in some countries as a topical cream (under the brand name Befar®), and preliminary studies have shown a clinical efficacy of up to 83%.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can he considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Using a novel slice based biotinylation developed in the inventors' labs, GLP-1 (100 nM) was shown to increase DAT cell surface expression in rat striatal slices. Striatal slices were exposed to either vehicle or GLP-1 for 20 min and level of DAT surface expression was evaluated using Western Blot of biotinylated brain tissue. GLP-1 treatment significantly increased surface expression of DAT, quantified as the ratio of surface to total DAT. GLP-1 did not affect surface expression of the Na+/K+ ATPase (FIG. 4B) suggesting that GLP-1 regulation of DAT surface levels is not due to a global effect on trafficking of plasma membrane proteins. In addition, the absence of tyrosine hydroxylase (TH, a cytosolic protein found in DAT terminals) from the biotinylated surface fraction serves a control for the integrity of our slice preparation and specificity of the surface signal.

### Example 2

Striatal slices were exposed to either vehicle or GLP-1 (100 nM) for 10 min and DAT activity was evaluated by radioalabed DA [³H-DA] uptake. Slices were exposed to ³H-DA for 10 min and then washed to remove excess DA at 4° C. to stop uptake. Slices were homogenized and ³H-DA quantified by a scintillation counter. GLP-1 significantly increased DA uptake.

### Example 3

Chronoamperometry was used to evaluate AMPH-induced DA efflux in striatal slices. AMPH (10 μM) was perfused onto the slices after pretreatment with either vehicle or GLP-1 (100 nM, for 20 min). Pretreatment with GLP-1 significantly potentiates the AMPH-induced increase in extracellular DA. This AMPH-induced increase in extracellular DA was cocaine sensitive suggesting it is mediated by DAT.

### Example 4

Locomotor activity was evaluated in the presence or absence of AMPH (2.78 mg/Kg, i.p.) in animals treated either with vehicle or GLP-1 agonist EX4 (30 μg/kg, i.p.) 90 min prior AMPH treatment. EX 4 treatment significantly decreased both basal locomotion (absence of AMPI-1) and AMPH-induced locomotor activity. This data demonstrate that GLP-1 signaling blunts AMPH-induced behavior.

### Example 5

Exenatide is supplied for SC injection as a sterile, preserved isotonic solution in a glass cartridge that has been assembled in a pen-injector (pen). Each milliliter (mL) contains 250 micrograms (mcg) synthetic exenatide, 2.2 mg metacresol as an antimicrobial preservative, mannitol as a tonicity-adjusting agent, and glacial acetic acid and sodium acetate trihydrate in water for injection as a buffering solution at pH 4.5. Two prefilled pens are available to deliver unit doses of 5 mcg or 10 mcg. Each prefilled pen will deliver 60 doses to provide for 30 days of twice daily administration (BID).

An individual addicted to amphetamine-like substances such as methamphetamine and/or drug of abuse acting on the dopaminergic system including cocaine will he administered GLP-1 and/or GLP-1 analogs to decrease the dependency on such drugs and to decrease the behaviors induced by these drugs.

An individual previously addicted to amphetamine-like substances such as methamphetamine and/or drug of abuse acting on the dopaminergic system including cocaine will he administered GLP-1 and/or GLP-1 analogs to decrease the probability of relapse.

An individual affected by neurophsychiatric disorders with a pathophysiology depending on dysregulation of dopamine signaling such as ADHD, schizophrenia, Bipolar disorder will be administered GLP-1 and/or GLP-1 analogs to restore normal dopamine homeostasis and dopamine transporter function.

### Example 6

In FIG. 2, rat tissue was ground up on dry ice and homogenized in lysis buffer containing SDS, separated by SDS-PAGE and the blot probed for GLP-1 R with an antibody to it (GLP-1R (H-55): sc-66911, Santa Cruz Biotechnologies). Lung, stomach and pancreas were used as positive controls. The overwhelming chemiluminesence signal achieved with penile tissue, which was more difficult to homogenize than the others, suggests that the receptor is present in high abundance in the rat penis.

In FIGS. 3 and 4A-B, the inventors sought to establish more specifically the localization of the GLP-1 receptor in the penile tissue. They performed both 40 micron longitudinal and transverse sections of the paraformaldehyde-perfused rat penis and stained for both GLP-1 receptor (green fluoresence) and nNOS (neuronal nitric oxide synthase) (red fluoresence). In FIG. 3, in the longitudinal section, the inventors observed abundant neuronal staining of the GLP-1 receptor in the corpora cavernosa. As seen in the zoomed in cavernosal nerve bundle (flanked by 2 purple lines) as well as the inset (yellow box), the GLP-1 receptor is being transported in the axons (green) and the DIC (white, black arrows) shows the myelin sheeth around the nerves (green). FIG. 4A also shows a longitudinal section, with GLP-1 receptor colocalazing with nNOS in the nerves (yellow). FIG. 4B shows a transverse section of the penis, where the receptor is in endothelial cells.

The inventors are conducting experiments to confirm that these are definitively endothelial cells by staining with eNOS in the same sections and looking for colocalization. Taking the data of FIGS. 2-3B together, two different techniques have shown the presence of the GLP-1 receptor in the penis for the first time.

All of the compositions and/or method's disclosed and claimed herein can he made and executed without undue experimentation in light of the present disclosure. while the compositions and methods of this invention have been described in terms of preferred embodiments, it will be apparent to those of skill in the art that variations may he applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### F. REFERENCES

The following references, to the extent that they provide exemplary procedural or other details supplementary to those set forth herein, are specifically incorporated herein by reference.
Bannon. Eur. Neuropsychopharmacol., 11(6):449-55, 2001.
Giros et al, Mol. Pharmacol., 42(3):383-390, 1992.
Kawarai et al., Gene, August 11; 195(1):11-8 1997.

1. A method of inhibiting a subject's response to an addictive substance comprising administering to said subject GLP-1 or analog thereof or an agonist of GLP-1 receptor.

2-4. (canceled)

5. The method of claim 1, wherein GLP-1 is administered.

6. The method of claim 1, wherein a GLP-1 receptor agonist is administered.

7. The method of claim 1, wherein a GLP-1 analog is administered.

8. The method of claim 1, wherein administering comprises oral administration, intravenous administration, transdermal administration, or inhalation.

9. The method of claim 1, wherein said addictive substance is alcohol, a narcotic, or a stimulant.

10-13. (canceled)

14. The method of claim 1, wherein said subject has been diagnosed with addictive behavior.

15. The method of claim 14, wherein said subject is currently using an addictive substance.

16. The method of claim 14, wherein said subject is not currently using an addictive substance.

17. The method of claim 14, wherein said subject further is receiving a second anti-addiction treatment.

18. A method of treating a subject for addiction, for reducing a subject's dependence on an addictive substance, for reducing a subject's chance of developing dependence on an addictive substance, or for modifying a subject's response to a drug acting through the dopamine receptor comprising administering to said subject GLP-1 or analog thereof or an agonist of GLP-1 receptor.

19-21. (canceled)

22. A method of treating a subject with an addictive substance comprising:
(a) administering to said subject GLP-1 or analog thereof or an agonist of GLP-1 receptor; and
(b) administering to said subject said addictive substance.

23-24. (canceled)

25. (canceled)

26. The method of claim 22, wherein GLP-1 is administered.

27. The method of claim 22, wherein a GLP-1 receptor agonist is administered.

28. The method of claim 22, wherein a GLP-1 analog is administered.

29. The method of claim 22, wherein administering comprises oral administration, intravenous administration, transdermal administration, or inhalation.

30. The method of claim 22, wherein said addictive substance is alcohol, a narcotic, or a stimulant.

31-34. (canceled)

35. A method of treating erectile dysfunction (ED) in a male subject comprising administering to said subject GLP-1 or analog thereof or an agonist of GLP-1 receptor.

36-37. (canceled)

38. The method of claim 35, wherein more than one GLP-1 receptor agonist is administered.

39. The method of claim 35, wherein GLP-1 is administered.

40. The method of claim 35, wherein a GLP-1 analog is administered.

41. The method of claim 35, wherein administering comprises oral administration, intravenous administration, transdermal administration, or inhalation.

42. The method of claim 35, wherein said subject has diabetes or has cardiovascular disease.

43. (canceled)

44. The method of claim 35, further comprising administering to said subject a second ED therapy.

45. A method of improving sexual performance in a male subject comprising administering to said subject GLP-1 or analog thereof or an agonist of GLP-1 receptor.

46. The method of claim 45, wherein more than one GLP-1 receptor agonist is administered.

47. The method of claim 45, wherein GLP-1 is administered.

48. The method of claim 45, wherein a GLP-1 analog is administered.

49. The method of claim 45, wherein administering comprises oral administration, intravenous administration, transdermal administration, or inhalation.

50. A method of reducing one or more symptoms of erectile dysfunction (ED) in a male subject comprising administering to said subject GLP-1 or analog thereof or an agonist of GLP-1 receptor.

51. The method of claim 50, wherein said one or more symptoms comprise partial erection, lack of erection, or limited erection duration.

52-54. (canceled)

55. (canceled)

56. A method of reducing one or more erectile dysfunction (ED) treatment side effects in a male subject comprising administering to said subject a reduced amount of ED treatment in combination with a GLP-1 or analog thereof or an agonist of GLP-1 receptor.

57. The method of claim 56, wherein said one or more treatment side effects comprise priapism, vision abnormalities, hearing loss, headache, muscle ache, and dyspepsia.

58-61. (canceled)

62. A method treating diabetes and erectile dysfunction (ED) in a male subject comprising administering to said subject an amount of a GLP-1R agonist effective to treat both diabetes and ED.

63-66. (canceled)
